(19) United States
(12) Patent Application Publication
(10) Pub. No.: US 2005/0182341 A1
(43) Pub. Date: Aug. 18, 2005

(54) Posture diagnosis equipment and program therefor

(76) Inventors: Ken Katayama, Nagasaki (JP); Kazuto Torii, Kyoto (JP); Naoji Akiyama, Osaka (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(21) Appl. No.: 10/822,869

(22) Filed: Apr. 13, 2004

(30) Foreign Application Priority Data
Feb. 13, 2004 (JP) P2004-37526

(51) Int. Cl. A61B 5/103; A61B 5/117

(52) U.S. Cl. 600/587; 600/592; 600/594

(57) ABSTRACT

To provide means capable of diagnosing a posture of an examinee conveniently, posture diagnosis equipment 1 is constructed including: photograph data receiving means 101 to receive photograph data obtained by photographing of a posture of an examinee from plural predetermined directions; photograph data display means 102 to display the photograph data received; foot pressure data receiving means 103 to receive foot pressure data obtained by measurement of the foot pressure of the examinee; gravitational center line calculating means 104 to calculate a gravitational center line which passes vertically through a gravitational center of the examinee based on the foot pressure data received; posture diagnosis point coordinates receiving means 105 to receive the coordinates of a posture diagnosis point specified relative to the photograph data displayed; and figure judgment means 106 to diagnose and typify the posture of the examinee based on the gravitational center line and the coordinates of the posture diagnosis point.

### TECHNICAL FIELD

The present invention relates to posture diagnosis equipment and posture diagnosis program for use in diagnosis of a figure of a human.

### BACKGROUND ART

Techniques as disclosed in the patent literature documents (Japanese Patent Laid-Open Publication No. 2002-213923, Japanese Patent Laid-Open Publication No. HEI 09-330424) for example are known as means for measuring a posture of a human body. These known techniques are of the type which irradiates an examinee attached with markers at different body parts with light wave and senses reflected light from the markers to determine the positions of the markers, thereby measuring a posture of the examinee's body.

Such a measuring device as described above, however, requires that: a large number of markers be attached to the body of the examinee and, what is more, be located at specific points of the body precisely; and conditions for lighting for irradiation of the markers and for environmental light be completed. For this reason, complicated and large-scale preparatory operations are required. Thus, the measuring device is not necessarily suitable for diagnosis of a posture of the body of each examinee in sports clubs, sports gyms, schools, medical institutions or the like.

The present invention made in view of the foregoing has an intended object to provide means capable of diagnosing a posture of an examinee conveniently.

### DISCLOSURE OF THE INVENTION

In order to solve the aforementioned problem, the present invention provides posture diagnosis equipment 1 capable of diagnosing a posture of an examinee for use in combination with a photographing device 2 for photographing the posture of the examinee and a foot pressure measuring device 3 for measuring a foot pressure of the examinee, as shown in FIG. 1, the posture diagnosis equipment 1 comprising: photograph data receiving means 101 to receive photograph data obtained by photographing of the posture of the examinee from plural predetermined directions with the photographing device 2; photograph data display means 102 to display the photograph data received by the photograph data receiving means 101; foot pressure data receiving means 103 to receive foot pressure data obtained by measurement of the foot pressure of the examinee with the foot pressure measuring device 3; gravitational center line calculating means 104 to calculate a gravitational center line of the examinee which passes vertically through a gravitational center of the examinee based on the foot pressure data received by the foot pressure data receiving means 103; posture diagnosis point coordinates receiving means 105 to receive the coordinates of a posture diagnosis point serving as an indicator for diagnosis of the posture of the examinee, the posture diagnosis point being specified by a diagnostician relative to the photograph data displayed by the photograph data display means 102; and figure judgment means 106 to diagnose and typify the posture of the examinee based on the gravitational center line calculated by the gravitational center line calculating means 104 and the coordinates of the posture diagnosis point received by the posture diagnosis point coordinates receiving means 105.

This equipment eliminates the troublesomeness that a large number of markers need be attached to the body of the examinee. Further, since the equipment receives an input of each posture diagnosis point specified on a photographed image instead of determining the position of each marker, the posture of the examinee can be measured with photographing device 2 of a common type. Moreover, there is no need to complete conditions for lighting and environmental light. Therefore, it becomes possible to diagnose a posture of each examinee in sports clubs, sports gyms, schools, medical institutions or the like conveniently. Based on such diagnosis, comprehensive evaluation becomes feasible which includes reference to the photograph data obtained by photographing of the examinee and the foot pressure data obtained by measurement of the foot pressure of the examinee.

As shown in FIG. 2, the posture diagnosis equipment 1 may further comprise averaging means 107 to sum up and then average plural photograph data items related to a single photographing direction received by the photograph data receiving means 101 to obtain a single photograph data item related to the single direction, wherein the photograph data display means 102 is operative to display the photograph data having undergone the averaging process by the averaging means 107. Such an arrangement can reduce noise of the photograph data thereby improving the image quality.

The posture diagnosis equipment 1 may further comprise sharpening means 108 to sharpen the photograph data, wherein the photograph data display means 102 is operative to display the photograph data having undergone the sharpening process by the sharpening means 108. Such an arrangement allows diagnosis of the posture of the examinee to be made more correctly.

As shown in FIG. 3, the posture diagnosis equipment 1 may further comprise inclination error correction means 109 to correct the photograph data to reduce an inclination error which is a degree of inclination of an upper edge of the foot pressure measuring device 3 appearing in the photograph data relative to a horizontal axis of the photograph data, wherein the photograph data display means 102 is operative to display the photograph data having undergone the correction process by the inclination error correction means 109. Such an arrangement is capable of reducing the inclination error which greatly affects the judgment on the figure of the examinee, i.e., an inclination of the photographing device 2 photographing the examinee, an inclination of the foot pressure measuring device 3 serving as a pedestal on which the examinee stands, or a like inclination.

The posture diagnosis equipment 1 may have an arrangement wherein: the foot pressure measuring device 3 has inclination measurement reference points as a reference for measurement of the inclination error at predetermined locations thereon; the photograph data to be received by the photograph data receiving means 101 contains inclination measurement reference point data on the inclination measurement reference points photographed; and the inclination error correction means 109 is operative to measure the inclination error based on the inclination measurement reference point data contained in the photograph data and then reduce the inclination error. Such an arrangement enables inclination error correction to be achieved with high precision. More specifically, this arrangement is such that: the foot pressure measuring device 3 has plural inclination measurement reference points located on a horizontal straight line on a surface thereof facing the photographing device 2; the photograph data to be received by the photograph data receiving means 101 contains inclination measurement reference point data on the plural inclination measurement reference points photographed; and the inclination error correction means 109 is operative to reduce the inclination error, regarding as the inclination error a degree of inclination of a straight line linking the plural inclination measurement reference points appearing in the photograph data relative to the horizontal axis of the photograph data.

The posture diagnosis equipment 1 may have an arrangement wherein: the inclination measurement reference points are operative to blink; the photograph data receiving means 101 is operative to receive a photograph data item obtained when the inclination measurement reference points are bright and a photograph data item obtained when the inclination measurement reference points are dark; and the inclination error correction means 109 is operative to detect the inclination measurement reference points from the difference between the photograph data items. Such an arrangement is preferable because it allows detection of the inclination measurement reference points to be achieved rapidly and accurately.

As shown in FIG. 4, the posture diagnosis equipment 1 may further comprise gravitational center line display means 110 to display the gravitational center line calculated by the gravitational center line calculating means 104 in a manner to superimpose the gravitational center line on the photograph data displayed by the photograph data display means 102. Such an arrangement will be of help to the diagnostician in specifying and inputting the posture diagnosis point, making diagnosis of the posture of the examinee, and like operations.

The posture diagnosis equipment 1 may further comprise: reference position determining means 111 to determine a reference position based on a position which is applied with pressure sensed by the foot pressure measuring device 3 when a reference member for use in determining the reference position is placed on the foot pressure measuring device 3; and center line display means to display a vertical center line passing through the reference position determined by the reference position determining means 111 in a manner to superimpose the center line on the photograph data displayed by the photograph data display means 102. Such an arrangement is capable of showing the difference between the center line and the gravitational center line to the diagnostician and the examinee.

If this arrangement further comprises horizontal distance calculating means 112 to calculate a horizontal distance between the center line and the gravitational center line, the posture diagnosis equipment 1 becomes capable of providing useful information for diagnosis of the posture of the examinee.

The posture diagnosis equipment 1 may have an arrangement wherein the photograph data display means 102 is capable of enlarged display of a region in the photograph data corresponding to a region around the posture diagnosis point when the posture diagnosis point coordinates receiving means 105 receives the coordinates of the posture diagnosis point. Such an arrangement can effectively lead the diagnostician to specify and input the posture diagnosis point and hence can raise the accuracy of inputting. Thus, it is possible to judge the posture more correctly.

The posture diagnosis equipment 1 may have an arrangement wherein the photograph data display means 102 is capable of displaying information indicative of the name and approximate location of the posture diagnosis point when the posture diagnosis point coordinates receiving means 105 receives the coordinates of the posture diagnosis point. Such an arrangement will be of help in specifying and inputting the posture diagnosis point. Even if the diagnostician is not very skilled, the arrangement allows the diagnostician to specify the posture diagnosis point.

The posture diagnosis equipment 1 may have an arrangement wherein the figure judgment means 106 is capable of visually outputting the posture of the examinee typified with use of a two-dimensional or three-dimensional model. Such an arrangement is capable of advantageously presenting the result of posture diagnosis to the diagnostician and the examinee. An embodiment of visualization of the posture of the examinee by the figure judgment means 106 includes categorizing the position of a body part intervening between correlated ones of posture diagnosis points into any one of a horizontal position, a vertical position, an inclined position (relative to the horizontal axis or vertical axis) and a twisted position (about the vertical axis) and then expressing the category clearly. This embodiment visualizes the positional relation between the correlated posture diagnosis points in a manner capable of judging whether or not the positional relation is in a preferable condition, i.e., whether or not the figure of the examinee is in a good condition. Another embodiment is configured to express clearly a stretched or contracted condition of body tissue, such as muscle, soft tissue, body cavity or other body tissue, intervening correlated ones of the posture diagnosis points. The “body tissue intervening between correlated ones of the posture diagnosis points”, as used here, means body tissue present in a region extending between plural posture diagnosis points and is not limited to muscle or like tissue directly interlinking one posture diagnosis point to another.

As shown in FIG. 5, the posture diagnosis equipment 1 may further comprise: foot pressure typifying means 113 to typify the foot pressure data received by the foot pressure data receiving means 103; and foot pressure pattern display means 114 to display a pattern of foot pressure typified by the foot pressure typifying means 113. This arrangement is preferable because the equipment becomes capable of making diagnosis taking the result of foot pressure measurement into consideration.

If the posture diagnosis equipment 1 further comprises advice information output means 115 to output information serving as advice about the posture of the examinee based on the posture of the examinee typified by the figure judgment means, the equipment 1 can contribute to improvement in the examinee's health.

According to the present invention, it is possible to diagnose a posture of an examinee conveniently.

### BRIEF DESCRIPTION OF THE DRAWINGS

FIG. 1 is a diagram illustrating a configuration of the present invention.

FIG. 2 is a diagram illustrating a configuration of the present invention.

FIG. 3 is a diagram illustrating a configuration of the present invention.

FIG. 4 is a diagram illustrating a configuration of the present invention.

FIG. 5 is a diagram illustrating a configuration of the present invention.

FIG. 6 is an illustration of a posture diagnosis system using posture diagnosis equipment according to the present invention.

FIG. 7 is an illustration of a variation of the system.

FIG. 8 is an illustration of a variation of the system.

FIG. 9 is an illustration of the relation between the foot pressure measuring device and the reference member in the system.

FIG. 10 is a diagram showing the hardware resources included in the posture diagnosis equipment.

FIG. 11 is a functional block diagram of the posture diagnosis equipment.

FIG. 12 is a representation for illustrating the sharpening process.

FIG. 13 is a representation for illustrating the inclination correction process.

FIG. 14 is a representation for illustrating the inclination correction process on the photograph data.

FIG. 15 is an illustration of an example of foot pressure data.

FIG. 16 is a representation for illustrating the gravitational center line calculating process.

FIG. 17 is an illustration of an exemplary display of the photograph data.

FIGS. 18A, 18B and 18C are illustrations of an exemplary display of the photograph data and name of the posture diagnosis point.

FIG. 19 is an illustration of an exemplary display of the foot pressure data.

FIGS. 20A, 20B, 20C, 20D, 20E and 20F are tables showing criteria for judgment of the figure of an examinee.

FIG. 21 is an illustration of an exemplary display of a model of the figure of the examinee.

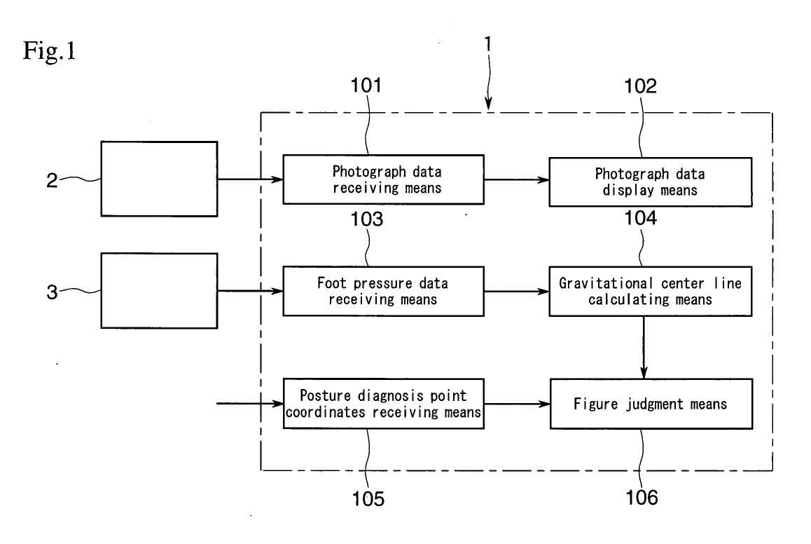

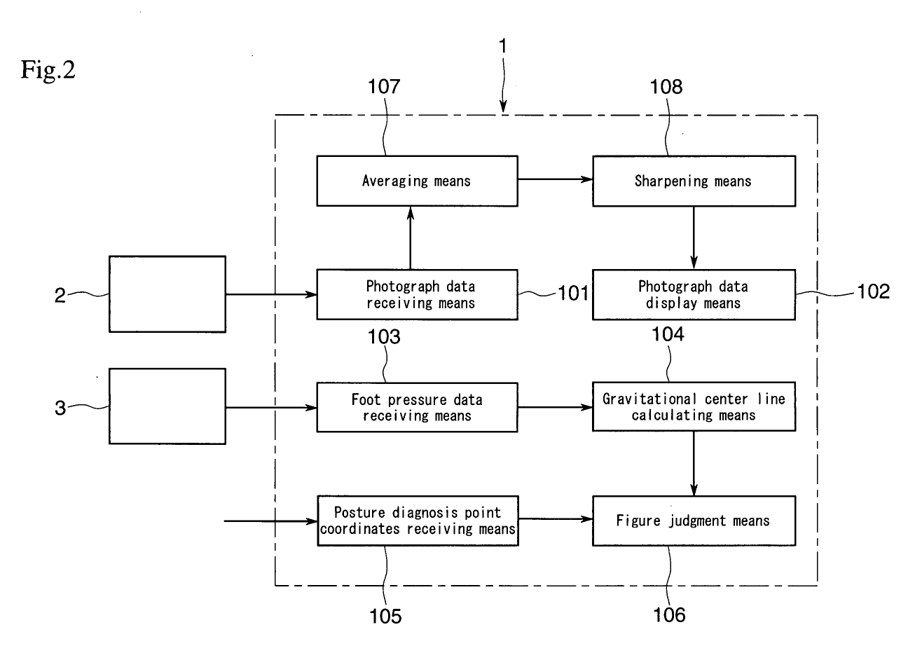

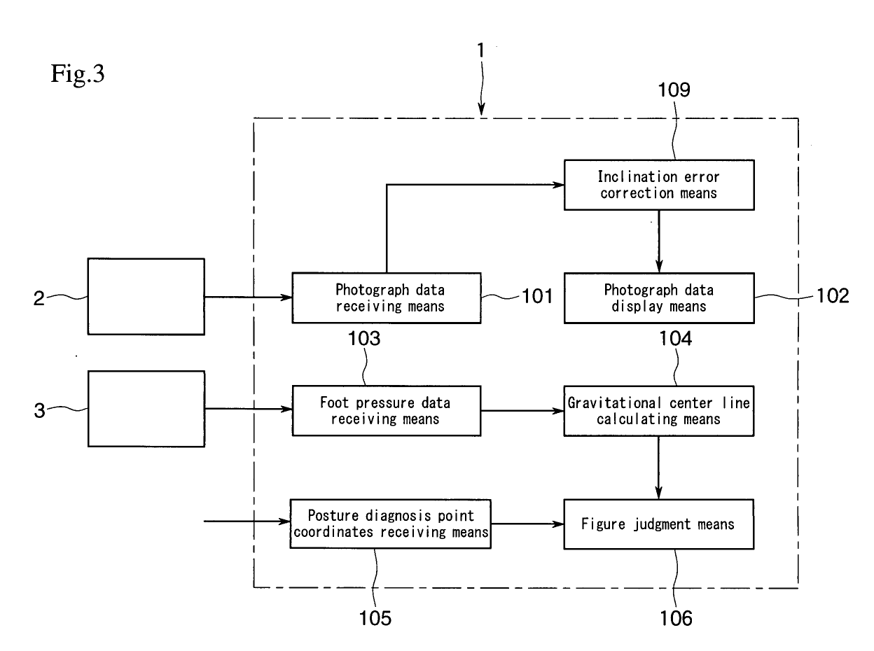

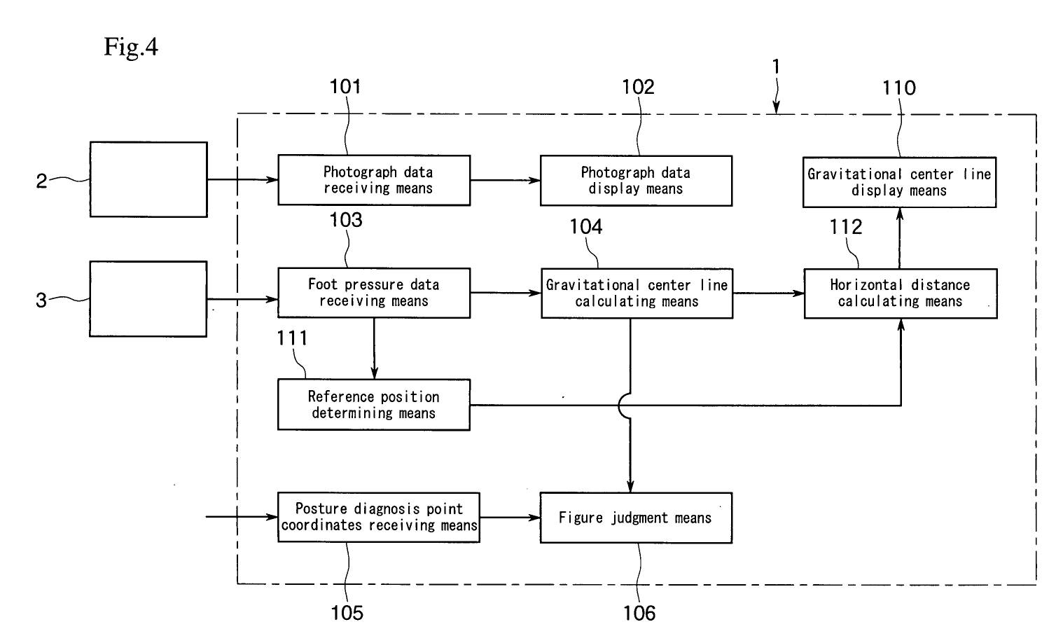

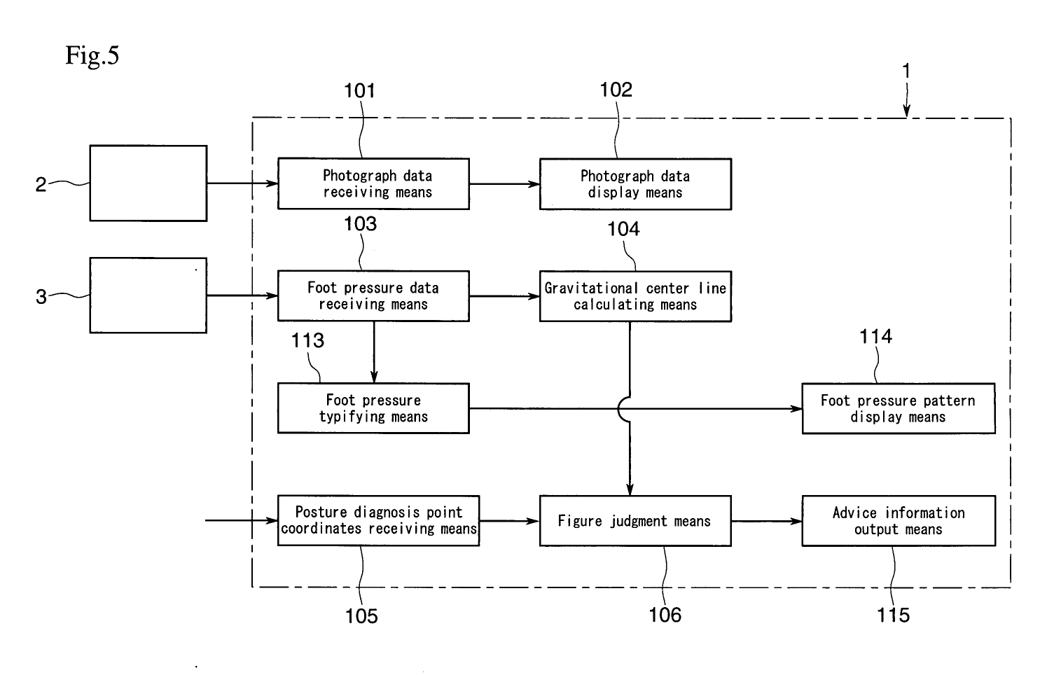

### BEST MODE FOR CARRYING OUT THE INVENTION

Hereinafter, one embodiment of the present invention will be described with reference to the drawings. A system according to this embodiment includes a photographing device 2, a foot pressure measuring device 3 and posture diagnosis equipment 1 as major constituents. The posture diagnosis equipment 1 is configured to diagnose a posture of an examinee based on a photograph of the examinee's posture taken with the photographing device 2 and a foot pressure of the examinee measured with the foot pressure measuring device 3.

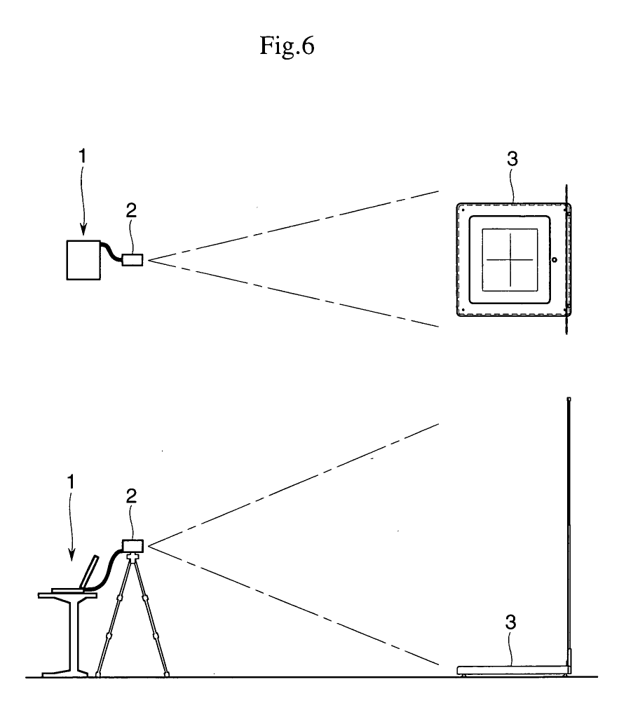

In the first place, the photographing device 2 and foot pressure measuring device 3 are described in brief. As shown in FIG. 6, the photographing device 2 is positioned to face the foot pressure measuring device 3 and photographs a posture of the examinee standing on the foot pressure measuring device 3. In this embodiment the photographing device 2 is a digital video camera capable of taking a motion picture having 30 frames per second and handling each frame of the motion picture taken by the video camera as a single still image. However, the photographing device 2 may be any device which can photograph the posture of the examinee and, hence, it is taken for granted that the photographing device 2 may employ a camera or the like which can take only a still image.

The foot pressure measuring device 3 is a known one having a flat pedestal shaped substantially rectangular in plan view and a pressure sensor shaped substantially square in plan view which is mounted on the upper surface of the pedestal. The foot pressure measuring device 3 is capable of measuring the foot pressures of the both feet of the examinee standing on the device 3 as stepping on the pressure sensor at a time. The foot pressure measuring device 3 may be one configured to determine a foot pressure distribution as well as the position of the gravitational center of the foot pressure distribution.

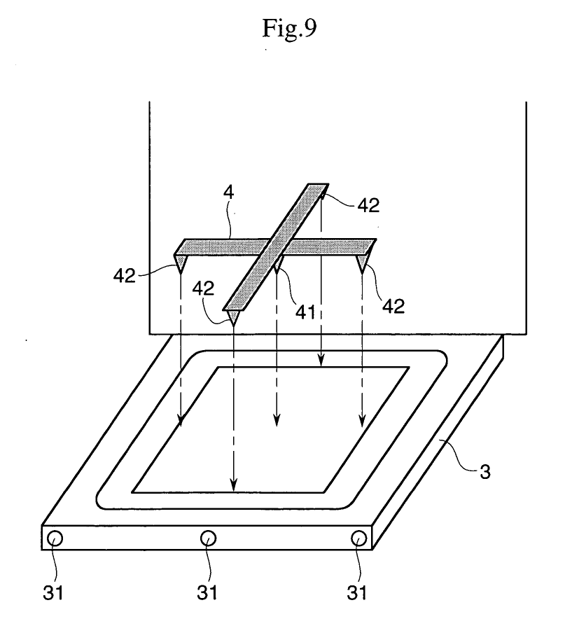

As schematically shown in FIG. 9 or a like figure, the foot pressure measuring device 3 further has a plurality of inclination measurement reference points 31 on at least one peripheral side of the pedestal. The inclination measurement reference points 31 are aligned on a horizontal straight line. In the example shown, three inclination measurement reference points 31 are equally spaced from each other. The center of the inclination measurement reference point located centrally of the row of the reference points 31 is positioned to coincide with the (breadthwise) central axis of the pressure sensor. The peripheral side of the pedestal, which is provided with the inclination measurement reference points 31, faces the photographing device 2. The photographing device 2 photographs the posture of the examinee together with the inclination measurement reference points 31. The inclination measurement reference points 31 serve as a reference for correction of an inclination error to be described later. This embodiment uses a light-emitting device such as a light-emitting diode for each of the inclination measurement reference points 31 and causes the inclination measurement reference points 31 to blink at predetermined time intervals.

In diagnosis of the posture of the examinee, the examinee needs to be photographed from plural directions so that the front side, lateral sides and rear side of the examinee are photographed. Usually, the photographing device 2 and the foot pressure measuring device 3 are fixed as opposed to each other with a predetermined spacing therebetween (and with a screen behind the foot pressure measuring device 3) as shown in FIG. 6 and the examinee is photographed from plural directions by having the examinee on the foot pressure measuring device 3 turn into front-facing position, side-facing position and rear-facing position with respect to the photographing device 2. At the same time with photographing, measurement of the foot pressure of the examinee can be conducted.

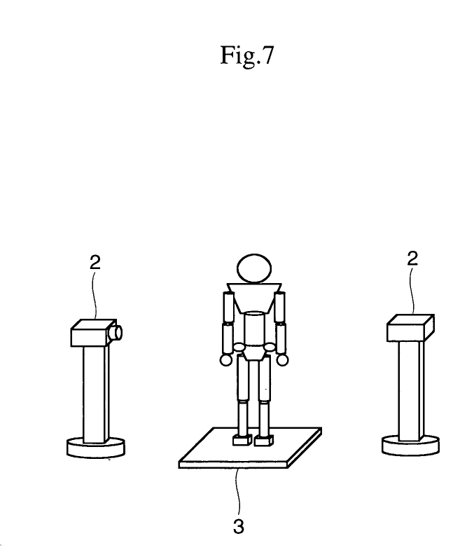

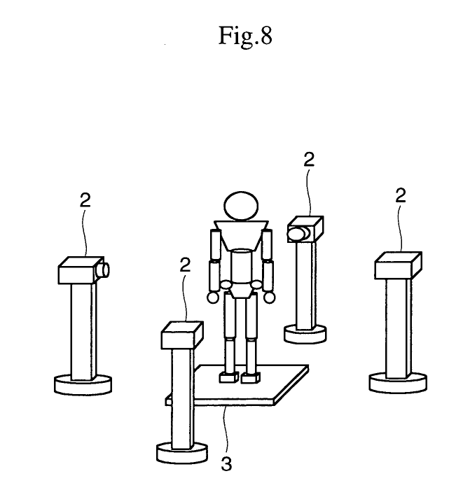

It is possible to use plural photographing devices 2 for the purpose of shortening the time required for photographing. In the case where two photographing devices 2 are used as shown in FIG. 7, the two peripheral sides of the foot pressure measuring device 3 which face respective of the photographing devices 2 need to be provided with a row of inclination measurement reference points 31 each, through the examinee can be photographed from two directions at a time without the need to change his or her position. Similarly, in the case where four photographing devices 2 are used as shown in FIG. 8, the four peripheral sides of the foot pressure measuring device 3 need to be provided with a row of inclination measurement reference points 31 each, through the examinee can be photographed from four directions at a time without the need to change his or her position.

This embodiment determines a reference position serving as a reference for foot pressure measurement prior to the measurement of the examinee's foot pressure. In determining the reference position, use is made of a reference member 4 as shown in FIG. 9. This reference member 4 is a thin plate member cross-shaped in plan view, having substantially conical projections 41 and 42 projecting downwardly from the center and four tips thereof. Reference member 4 not having the central projection 41 may be used. The reference member 4 is sized to have length and breadth substantially equal to those of the pressure sensor of the foot pressure measuring device 3. The reference position can be determined by measuring the pressure exerted on the pressure sensor from each projection of the reference member 4 placed on the foot pressure measuring device 3.

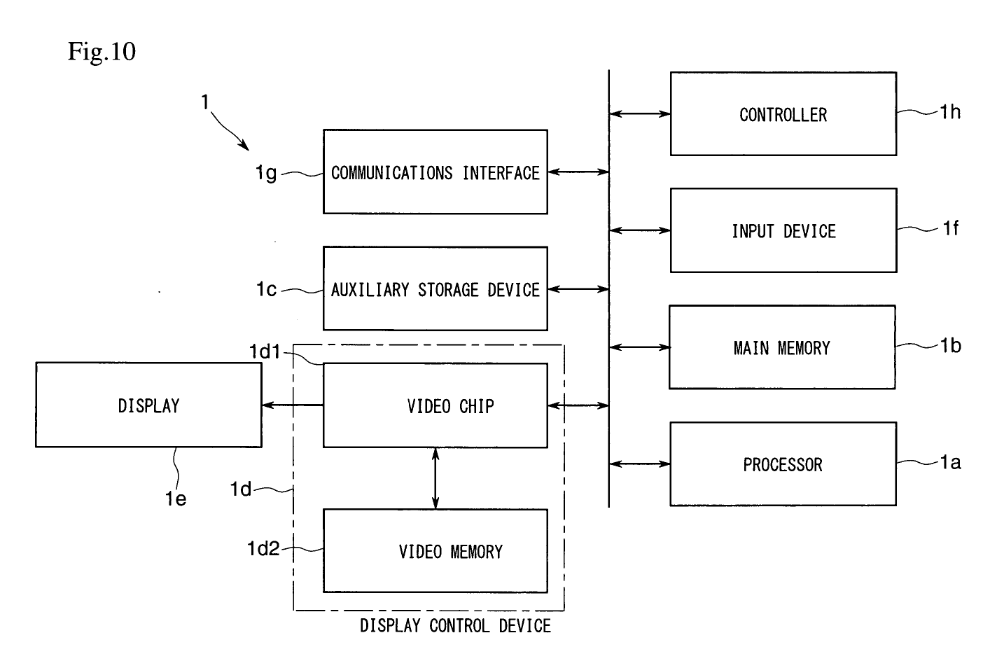

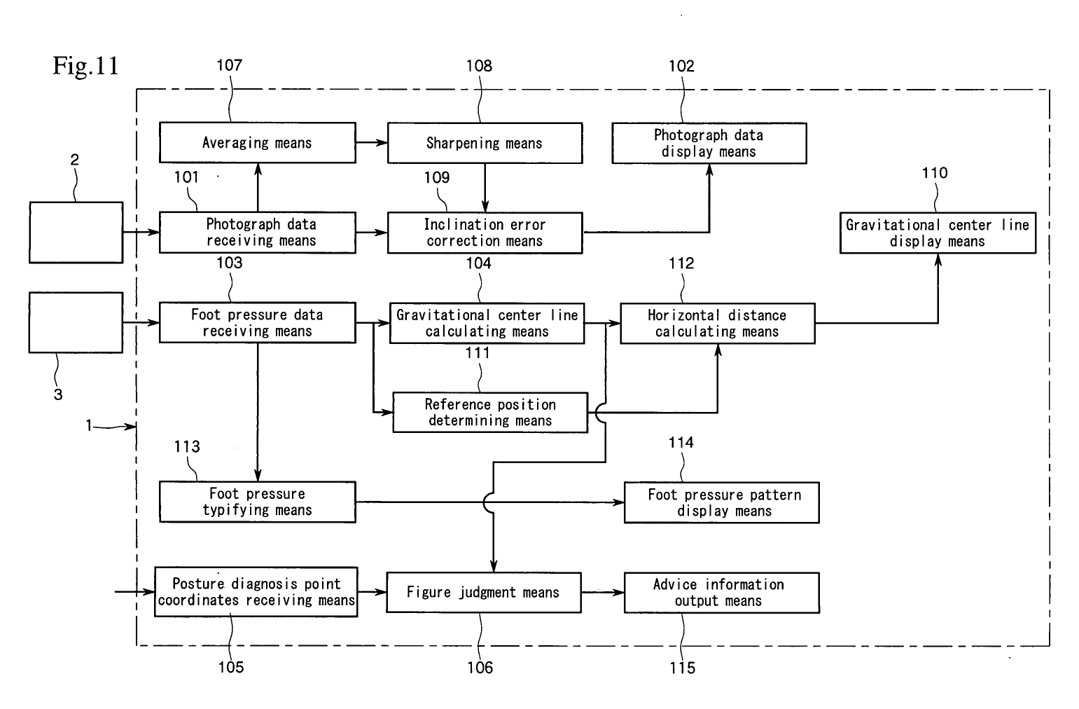

Detailed description will be made of the posture diagnosis equipment 1. The posture diagnosis equipment 1 has a predetermined image processing function and other general information processing function. As shown in FIG. 10, the posture diagnosis equipment 1 according to this embodiment includes, as a major component, a computer comprising hardware resources, such as processor 1a, main memory 1b, auxiliary storage device 1c, display control device 1d, display 1e, input device 1f and communications interface 1g, which are controlled for cooperation by controller 1h (system controller, I/O controller or the like). The auxiliary storage device 1c is a hard disk drive, an optical disk drive like DVD-ROM, or other drive, but may be a removable one, for example, a flash memory card or an external disk drive. The display control device 1d comprises such devices as a video chip (or graphics chip) 1d1 having a function to generate an image to be displayed according to an imaging instruction received from the processor 1a and then output the image to the display 1d, and video memory (Video RAM) 1d2 playing the role of temporarily storing image and the like. The input device 1f generally includes pushbuttons and keyboard, which can be manipulated with user's fingers, and pointing devices such as a mouse, track pad and touch panel. The communications interface 1g is an interface for transmitting and receiving a variety of data, for example, NIC (Network Interface Card), wireless LAN (Local Area Network) transceiver, USB (Universal Serial Bus), or IEEE 1394. The aforementioned photographing device 2 and foot pressure measuring device 3 are connected to the posture diagnosis equipment 1 via respective communications interfaces 1g.

Usually, the program to be executed by the processor 1a is stored in the auxiliary storage device 1c. When the program is to be executed, the program is loaded into the main memory 1b. from the auxiliary storage device 1c and then interpreted by the processor 1a. In the posture diagnosis equipment 1 of this embodiment are installed a known GUI (Graphical User Interface)-type OS (Operating System) program and various device driver programs accompanying the OS program, which intermediate between the aforementioned hardware resources utilized by application programs. A posture diagnosis program is installed over the aforementioned programs. According to the posture diagnosis program, the equipment 1 causes the hardware resources to operate, thereby exercising functions as photograph data receiving means 101, averaging means 107, sharpening means 108, inclination error correction means 109, foot pressure data receiving means 103, reference position determining means 111, gravitational center line calculating means 104, horizontal distance calculating means 112, photograph data display means 102, center line display means, gravitational center line display means 110, posture diagnosis point coordinates receiving means 105, figure judgment means 106, foot pressure typifying means 113, foot pressure pattern display means 114, and advice information output means 115.

Description will be made of each part. The photograph data receiving means 101 receives a motion picture or still image of a posture of the examinee taken from predetermined plural directions with the photographing device 2. Specifically, according to the program, the processor 1a fetches the photograph data from the photographing device 2 connected thereto through the communications interface 1g and stores the photograph data into a predetermined storage area of the main memory or auxiliary storage device 1c. However, the present invention does not preclude such an embodiment as to record the motion picture or still image taken by the photographing device 2 on a recording medium such as an optical disk or a video tape and then cause the posture diagnosis equipment 1 to read the motion picture or still image recorded on the recording medium. In this embodiment there are four photographing directions: front-side angle from which the front side of the examinee is photographed, left-side angle from which the left side of the examinee is photographed, right-side angle from which the right side of the examinee is photographed, and rear-side angle from which the rear side of the examinee is photographed. Photograph data items obtained by photographing from these angles will be referred to as “front-side photograph data”, “left-side photograph data”, “right-side photograph data” and “rear-side photograph data”, respectively. As already described, the photographing device 2 is capable of taking a motion picture. The photograph data receiving means 101 receives plural frames contained in a motion picture taken from a direction as photograph data items.

The averaging means 107 sums up and then averages plural photograph data items (i.e., frames) related to a single photographing direction which are received by the photograph data receiving means 101 into a single photograph data item related to the single direction. Specifically, the processor 1a performs a process of summing up and averaging the pixel values of respective photograph data items according to the program. Such an averaging process is performed on each of the front-side photograph data, left-side photograph data, right-side photograph data and rear-side photograph data. More specifically, the values obtained by addition of pixel values (R, G, B) of pixels located on a same point of coordinates (x, y) in plural photograph data items are divided by the number of photograph data items added together. In the case of averaging of n frames, assume that:
first frame (x, y)=(r1, g1, b1)
second frame (x, y)=(r2, g2, b2)
. . .
. . .
. . .
n-th frame (x, y)=(rn, gn, bn), then the pixel value (R, G, B) of the pixel on the point of the coordinates (x, y) in the photograph data resulting from the averaging process is given as follows:
   R=(r1+r2+ . . . +rn)/n
   G=(g1+g2+ . . . +gn)/n
   B=(b1+b2+ . . . +bn)/n.
   The averaging process makes it possible to reduce white noise contained in the photograph data. The averaging process need not necessarily be performed on all the pixels of the photograph data. It is conceivable to shorten the processing time by excluding a region where the examinee or the foot pressure measuring device 3 is apparently not seen from the object to be averaged.

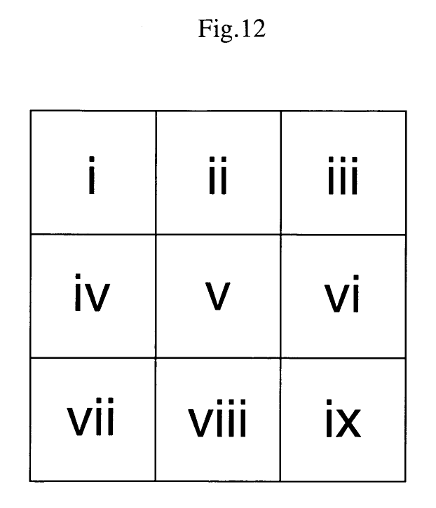

The sharpening means 108 sharpens the photograph data by a gray-scale process. Specifically, according to the program, the processor 1e performs a filtering process on the photograph data having undergone the averaging process. The sharpening process, also, is performed on each of the front-side photograph data, left-side photograph data, right-side photograph data and rear-side photograph data. An example of such a sharpening process to be performed by the sharpening means 108 will be described with reference to FIG. 12. In this figure, (i) to (ix) each indicate one pixel. When the pixel (v) is a direct target to be sharpened, the pixel value of each of the pixels (i), (ii), (iii), (iv), (vi), (vii), (viii) and (ix) present around the pixel (v) is multiplied by a factor of −0.25, whereas the pixel value of the target pixel (v) multiplied by a factor of 2.9. In this embodiment the factor by which the pixel value of the pixel (v) is multiplied is set smaller than 3, which is conventionally used. Thereafter, these pixel values are summed up. Assume that the pixel values of respective pixels are:
(i)=(R1, G1, B1)
(ii)=(R2, G2, B2)
. . .
. . .
. . .
(ix)=(R9, G9, B9), then the pixel value (R, G, B) of the pixel (v) having undergone the sharpening process is given as follows:
   R=R5*2.9+(R1+R2+R3+R4+R6+R7+R8+R9)*(−0.25)
   G=G5*2.9+(G1+G2+G3+G4+G6+G7+G8+G9)*(−0.25)
   B=B5*2.9+(B1+B2+B3+B4+B6+B7+B8+B9)*(−0.25).
   The sharpening process sharpens the examinee's posture appearing in the photograph data. It is conceivable to shorten the processing time by excluding a region where the examinee or the foot pressure measuring device 3 is apparently not seen from the object to be sharpened.

The inclination error correction means 109 corrects an inclination error which occurs due to the manner of photographing. The inclination error, which is a degree of inclination of an upper edge of the foot pressure measuring device 3 appearing in the photograph data relative to the horizontal axis of the photograph data, affects.diagnosis of the examinee's posture. Specifically, according to the program, the processor 1a determines the magnitude of the inclination error of the photograph data and performs a process of rotating the photograph data in such a direction as to reduce the inclination error. This correction process, also, is performed on each of the front-side photograph data, left-side photograph data, right-side photograph data and rear-side photograph data.

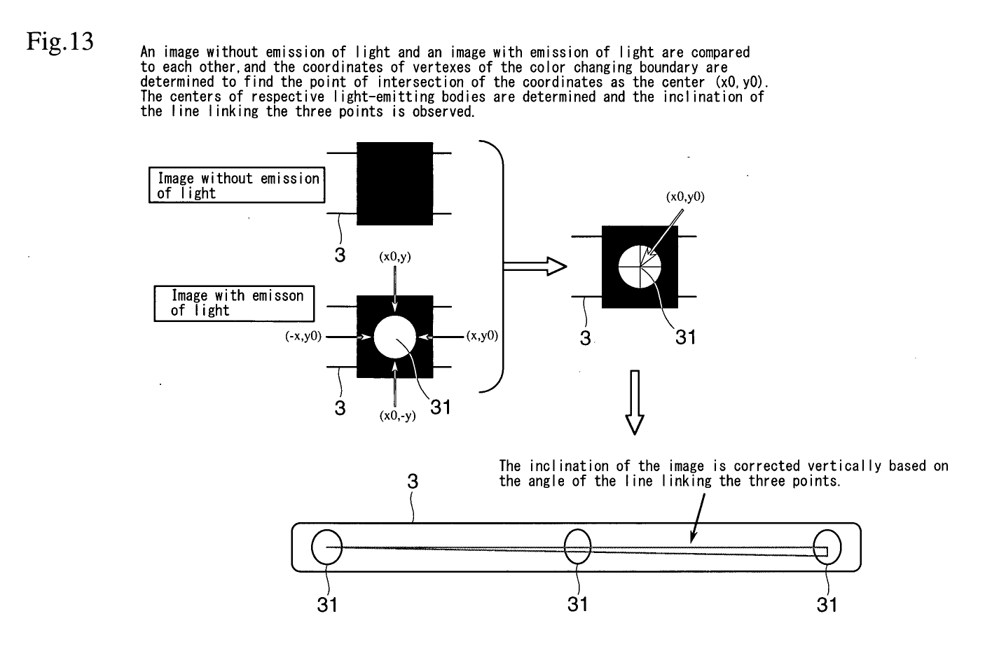

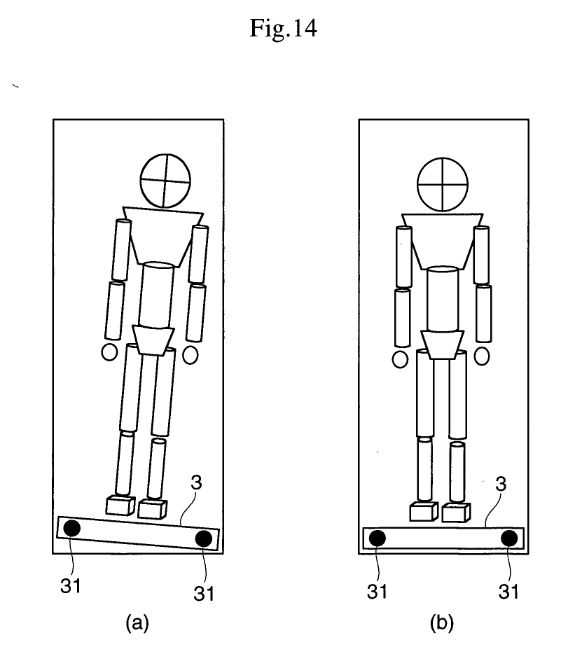

Detailed description will be made of the inclination correction procedure according to the program. As already described, the photographing device 2 photographs the examinee together with the inclination measurement reference points 31 provided on the foot pressure measuring device 3. Therefore, the photograph data to be received by the photograph data receiving means 101 contains data on the inclination measurement reference points 31 provided on the foot pressure measuring device 3. The inclination measurement reference points 31 are each configured to blink, while the photographing device 2 adapted to take a motion picture with the inclination measurement reference points 31 blinking. Accordingly, the photograph data receiving means 101 receives a photograph data item (frame) obtained when the inclination measurement reference points 31 are bright and a photograph data item obtained when the inclination measurement reference points 31 are dark. As shown in FIG. 13, the inclination error correction means 109 turns the pixel value of a region presumed to contain each inclination measurement reference point 31 into binary digits with use of a predetermined threshold value for each of the photograph data item (not averaged) obtained when the inclination measurement reference points 31 are bright and the photograph data item obtained when the inclination measurement reference points 31 are dark. This threshold value is set to a value distinguishing each inclination measurement reference point 31 in the brightly light-emitting state from the rest. The inclination measurement reference points 31 are detected from the difference between the photograph data item obtained when the inclination measurement reference points 31 are bright and the photograph data item obtained when the inclination measurement reference points 31 are dark. More specifically, the coordinates of each of vertical and horizontal vertexes of the boundary of a region having varying pixel value (i.e., a region corresponding to each inclination measurement reference point 31) are determined and then the coordinates of the point of intersection of the line segment linking the coordinates of respective vertical (upper and lower) vertexes and the line segment linking the coordinates of respective horizontal (right-hand and left-hand) vertexes are determined. This point of intersection is considered to be the center of each inclination measurement reference point 31. After having determined the coordinates of the respective centers of the plural inclination measurement reference points 31, the inclination of the straight line passing through the centers is found to obtain the inclination error. When the straight line is inclined, that is, when the straight line is not parallel with the horizontal axis of the photograph data, the angle formed between the straight line and the horizontal axis is the inclination error. Subsequently, as shown in FIG. 14, processing is performed to rotate the photograph data having undergone the averaging process (FIG. 14(a)) by the inclination error thus obtained, thereby obtaining final photograph data not containing the inclination error (FIG. 14(b)).

Meanwhile, if the ratio of the distance between (the centers of) the plural inclination measurement reference points 31 in the photograph data to the real distance therebetween on the real foot pressure measuring device 3 is previously known, it is possible to convert the distance between a certain pixel and another pixel in the photograph data into a real distance in a real space (scene photographed). In reverse, it is also possible to convert a real distance in the photographed scene into a corresponding distance in the photograph data. In this embodiment, data on the distance between (the centers of) real inclination measurement reference points 31 is previously stored in the main memory 1b or the auxiliary storage device 1c. Thus, the processor 1a is configured to calculate the ratio of the distance between (the centers of) the plural inclination measurement reference points 31 in the photograph data to the real distance therebetween in parallel with the above-described inclination correction process according to the program.

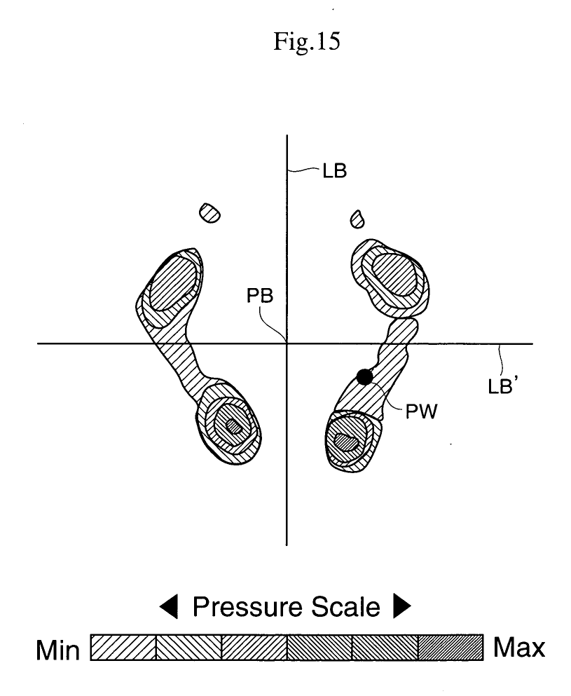

The foot pressure data receiving means 103 receives foot pressure data representing. the foot pressure distribution of the examinee measured by the foot pressure measuring device 3. Specifically, according to the program, the processor 1a fetches foot pressure data from the foot pressure measuring device 3 connected thereto through the communications interface 1g and stores the foot pressure data into a predetermined storage area of the main memory 1b or auxiliary storage device 1c. FIG. 15 shows an example of foot pressure data. The foot pressure data is, for example, in the form of a foot pressure picture (image) in which the magnitude of pressure at each location is reflected in the pixel value of a pixel at the same location. In the case where the foot pressure measuring device 3 is configured to measure a foot pressure distribution as well as to determine the position of the gravitational center of the foot pressure distribution, the foot pressure data to be received by the foot pressure data receiving means 103 contains data on the position of the gravitational center. However, the present invention does not preclude such an embodiment as to record the foot pressure data obtained from measurement by the foot pressure measuring device 3 on a recording medium such as an optical disk or flash memory and then cause the posture diagnosis equipment 1 to read the foot pressure data recorded on the recording medium.

The reference position determining means 111 determines a reference position for foot pressure measurement. The reference position determination is conducted prior to the measurement of the examinee's foot pressure. In the reference position determination the reference member 4 described earlier is used. As shown in FIG. 9, the reference member 4 is placed on the foot pressure measuring device 3 in a manner to position the front, rear, right and left projections 42 of the reference member 4 on the fore-and-aft center axis and the breadthwise center axis of the pressure sensor in plan view. The foot pressure measuring device 3 measures the pressures exerted through the projections 41 and 42. The measured pressure data obtained indicates the positions of the respective projections 41 and 42 of the reference member 4. According to the program, the processor 1a fetches the measured pressure data through the communications interface 1g and determines the reference position based on the measured data thus fetched. Specifically, the position of the projection 41 located centrally of the reference member 4 serves as the reference position PB for foot pressure measurement, while the line segment linking the right and left projections and the line segment linking the upper and lower (fore and aft) projections serve as reference lines LB and LB′ for foot pressure measurement. The position PB of the central projection 41 substantially coincides with the point of intersection of the reference lines LB and LB′. However, the reference member 4 may not have the central projection 41. In such a case the positions of respective four projections 42 are detected and the position of the point of intersection of the line segments each linking opposite projections 42 is determined as the reference position PB.

Meanwhile, if the ratio of the distance between plural projections 41,42 in the foot pressure data to the real distance therebetween on the real reference member 4 is previously known, it is possible to convert the distance between a certain pixel and another pixel in the foot pressure data into a real distance in a real space. In reverse, it is also possible to convert a real distance on the foot pressure measuring device 3 into a corresponding distance in the foot pressure data. In this embodiment, data on the distance between (the centers of) projections 41,42 of the reference member 4 is previously stored in the main memory 1b or the auxiliary storage device 1c. Thus, according to the program, the processor 1a is configured to calculate the ratio of the distance between plural projections 41,42 in the foot pressure data to the real distance therebetween in parallel with the above-described reference position determining process.

The gravitational center line calculating means 104 calculates the gravitational center line LW vertically passing through the gravitational center of the examinee based on the foot pressure data received by the foot pressure data receiving means 103. Specifically, the processor 1a calculates the vertical line passing through the gravitational center PW of the foot pressure distribution. In the case where the foot pressure data received by the foot pressure data receiving means 103 contains data on the position of the gravitational center, such data can be utilized. On the other hand, in the case where the foot pressure data does not contain the data on the position of the gravitational center, the gravitational center line calculating means 104 calculates first the gravitational center of the foot pressure distribution and then the gravitational center line LW.

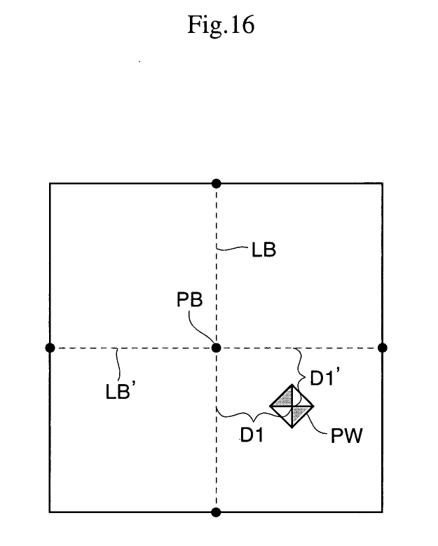

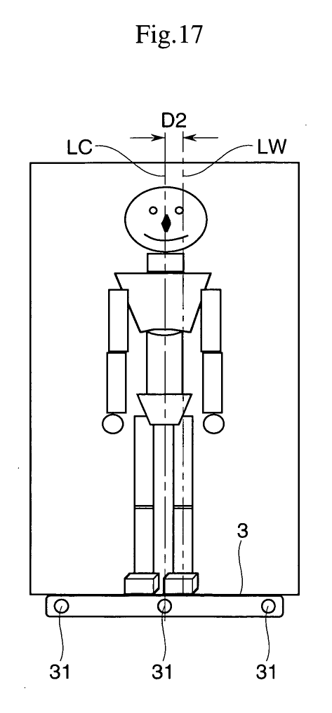

The horizontal distance calculating means 112 calculates the horizontal distance between the gravitational center line LW calculated by the gravitational center line calculating means 104 and the center line LC in the photograph data. Stated otherwise, the horizontal distance calculating means 112 determines the position of the gravitational center line LW in the photograph data. As already described, the ratio of the distance between pixels in the foot pressure data to the corresponding distance in a real space has been found by the reference position determining means 111, while the ratio of the distance between pixels in the photograph data to the corresponding distance in a real space has been found by the inclination error correction means 109. Further, since the foot pressure measuring device 3 is configured so that the breadthwise center axis of the pressure sensor coincides with the breadthwise center axis of the central inclination measurement reference point (that is, these center axes lie in the same vertical plane), the reference line LB dividing the foot pressure data image into a right-hand half and a left-hand half coincides with the center line LC dividing the photograph data into a right-hand half and a left-hand half. Therefore, it is possible to convert the position of the gravitational center in the foot pressure data into the position of the gravitational center in each of the front-side photograph data, left-side photograph data, right-side photograph data and rear-side photograph data. Referring to FIGS. 16 and 17, according to the program, the processor 1a determines how much (by how many pixels) the coordinates of the gravitational center PW in the foot pressure data are spaced apart from the breadthwise reference line PB (or lengthwise reference line PB) and then multiplies the determined distance D1 or D1′ by the ratio of the distance between corresponding pixels in the foot pressure data to the corresponding distance in the real space to find the real distance in the real space. Then, the processor 1a multiplies the real distance in the real space by the reciprocal of the ratio of the distance between the corresponding pixels in the photograph data to the corresponding distance in the real space to find distance D2. The distance D2 represents how much (by how many pixels) the center line LC and the gravitational center line LW are spaced from each other in each photograph data item.

The photograph data display means 102 displays the final front-side photograph data, left-side photograph data, right-side photograph data and rear-side photograph data having been received by the photograph data receiving means 101 and subjected to the averaging process, sharpening process and inclination correction process. Specifically, the processor 1a causes the display 1e to display the final photograph data on the screen according to the program. FIG. 17 shows an example of the photograph data displayed. In the example shown, the left-side photograph data and the right-side photograph data are collectively shown as right-and-left-side photograph data for convenience of description. The photograph data may be outputted as a hard copy from a printer (not shown).

The center line display means displays the vertical center line LC passing through the reference position determined by the reference position determining means 111 in a manner to superimpose the center line LC on the photograph data displayed by the photograph data display means 102. Specifically, the processor 1a further plots the center line LC on the photograph data displayed by the display 1e according to the program.

The gravitational center line display means 110 displays the gravitational center line LW of the examinee's foot pressure in a manner to superimpose the gravitational center line LW on the photograph data displayed by the photograph data display means 102. Specifically, the processor 1a further plots the gravitational center line LW on the photograph data displayed by the display 1e according to the program. At this time, the position of the gravitational center line LW is calculated by the above-described horizontal distance calculating means 112.

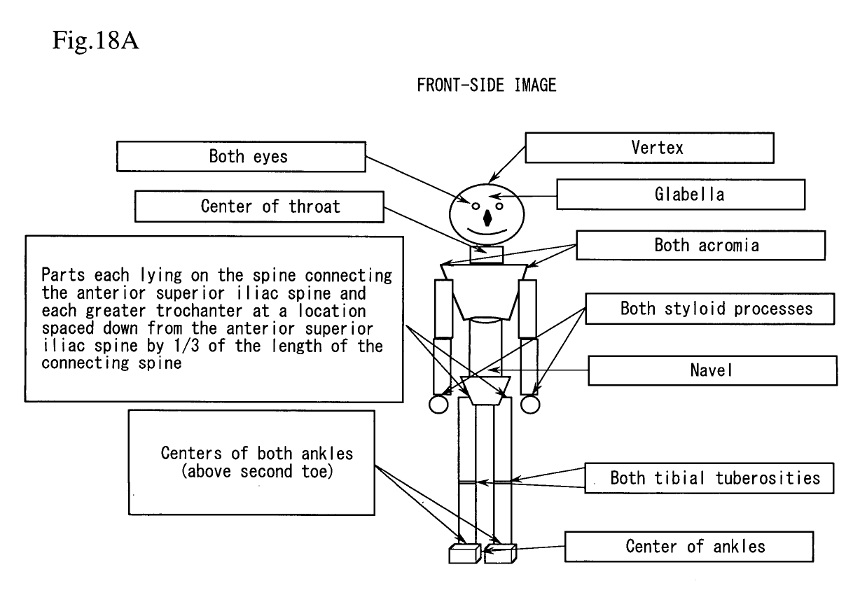

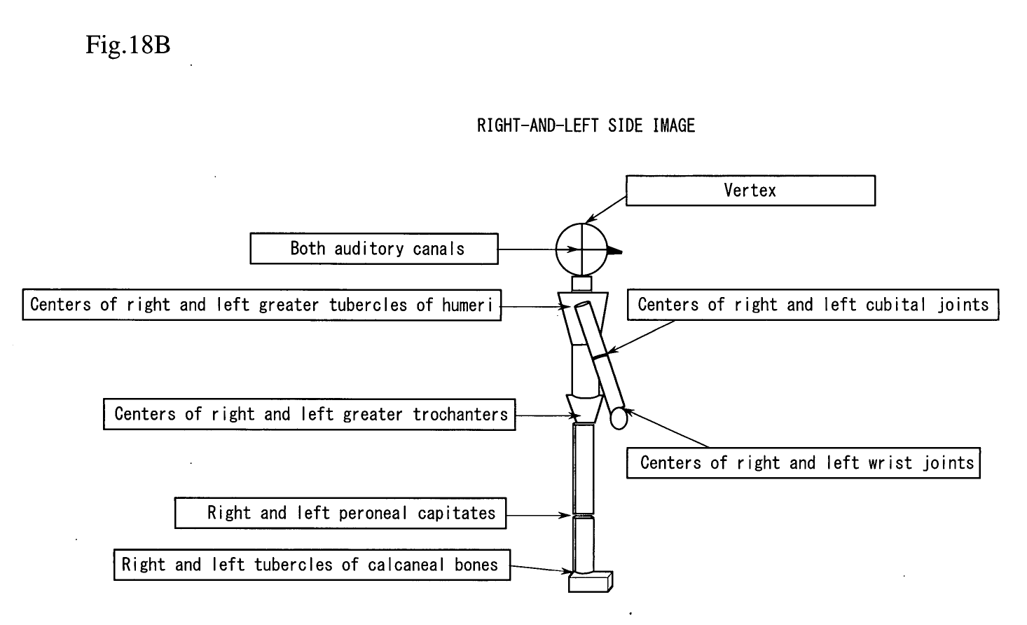

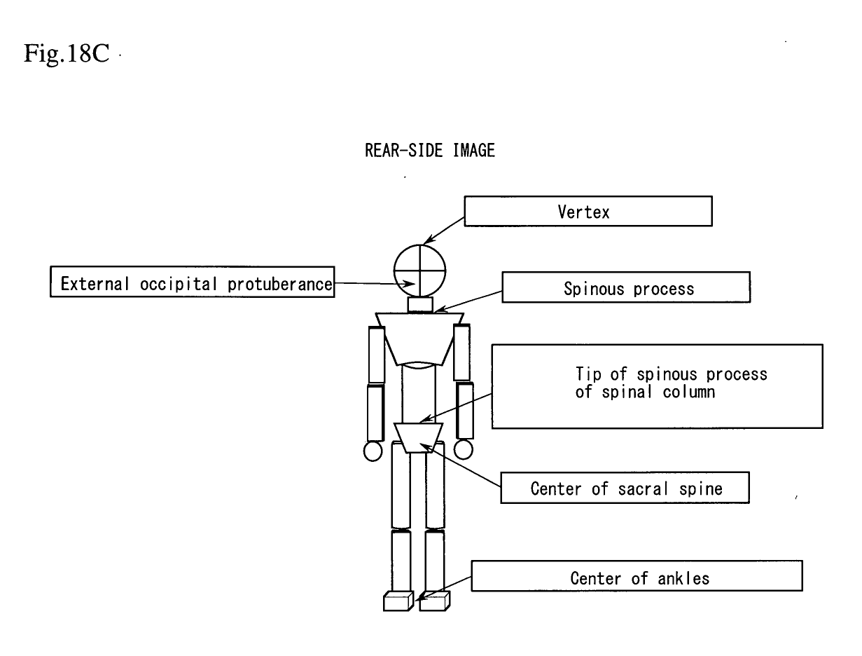

The posture diagnosis point coordinates receiving means 105 receives input of a posture diagnosis point specified relative to the photograph data displayed by the photograph data display means 102. The “posture diagnosis point” means a body part of the examinee serving as an indicator for diagnosis of the examinee's posture. FIGS. 18A, 18B and 18C show examples of such posture diagnosis points. The posture diagnosis point is specified manually by the diagnostician. Specifically, the processor 1a receives an input that is made by the diagnostician to specify the coordinates of the posture diagnosis point via the input device 1f according to the program. Usually, the diagnostician specifies each posture diagnosis point in a manner to specify a location in the photograph data displayed on the screen of the display 1e with use of a pointing device. The posture diagnosis point coordinates receiving means 105 receives such an input made by manipulation and stores the value of the coordinates of the posture diagnosis point in the photograph data into the main memory 1b or the auxiliary storage device 1c together with a related identifier for identifying the posture diagnosis point. For example, data on the coordinates of a posture diagnosis point “right eye” of the examinee is made related to an identifier for identifying the “right eye” and then stored in the main memory 1b or the auxiliary storage device 1c.

In this embodiment the photograph data display means 102 is configured to present information indicative of the name and approximate position of the posture diagnosis point to the diagnostician when the posture diagnosis point coordinates receiving means 105 receives an input specifying the posture diagnosis point. The “name of a posture diagnosis point”, as used here, is the name of a body part such as the vertex, both eyes, glabella, right and left auditory canals, or both acromia. The “information indicative of the approximate position of the posture diagnosis point” is, for example, an arrow as shown in FIGS. 18A, 18B and 18C. Further, the photograph data display means 102 is configured to be capable of enlargedly displaying a partial region of the photograph data which is expected to include an individual posture diagnosis point in the same window as displaying the same photograph data or another window when the posture diagnosis point coordinates receiving means 105 receives an input specifying the posture diagnosis point.

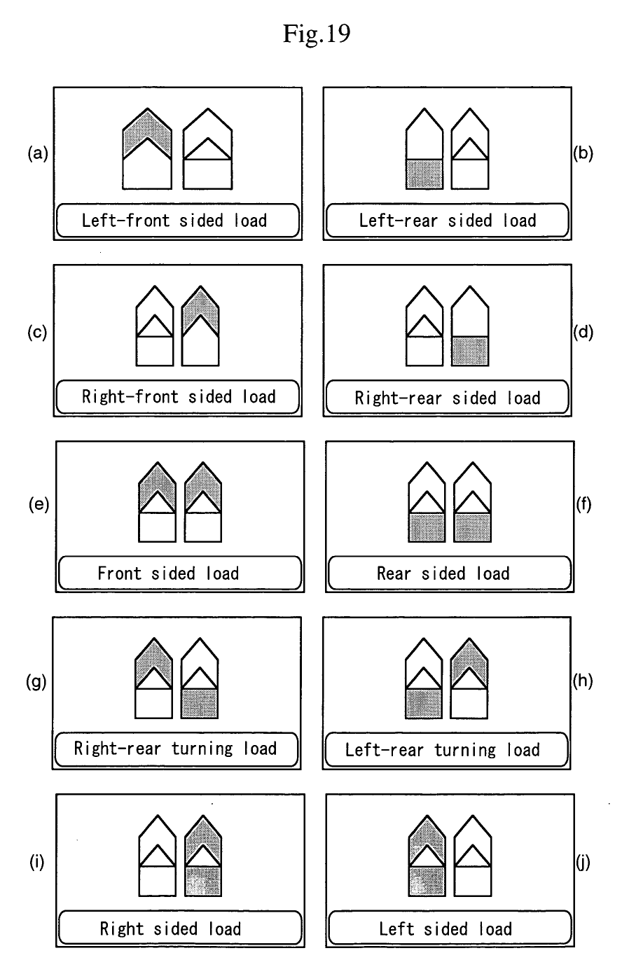

The foot pressure typifying means 113 typifies the foot pressure data received by the foot pressure data receiving means 103. Specifically, according to the program, the processor 1a typifies the foot pressure data by combining loads, such as a front sided load, rear sided load, left sided foot load and right sided foot load, determined based on the foot pressure data. FIG. 19 shows examples of types of foot pressure data. Here, the front sided load and the rear sided load are determined from the ratio between the front foot pressure and the rear foot pressure of each foot (or both feet.) The case where the ratio of the front (or rear) load distribution to the overall foot pressure exceeds a predetermined threshold value (for example 55%) is determined as the front (or rear) sided load, while the case where the ratio does not exceed the threshold value determined as equal load. Similarly, the case where the ratio of the right (or left) foot load distribution to the overall foot pressure of the both feet exceeds a predetermined threshold value is determined as the right (or left) sided foot load, while the case where the ratio does not exceed the threshold value determined as equal load.

The foot pressure pattern display means 114 displays the foot pressure data received by the foot pressure data receiving means 103 and a foot pressure pattern as a result of typification by the foot pressure typifying means 113. Specifically, the processor 1a causes the display 1e to display the foot pressure data and the category of the foot pressure pattern of the foot pressure data on its screen according to the program. FIGS. 19(a) to 19(j) show examples of foot pressure data and foot pressure pattern displayed. It is to be noted that the foot pressure data and foot pressure pattern may be outputted as a hard copy from the printer.

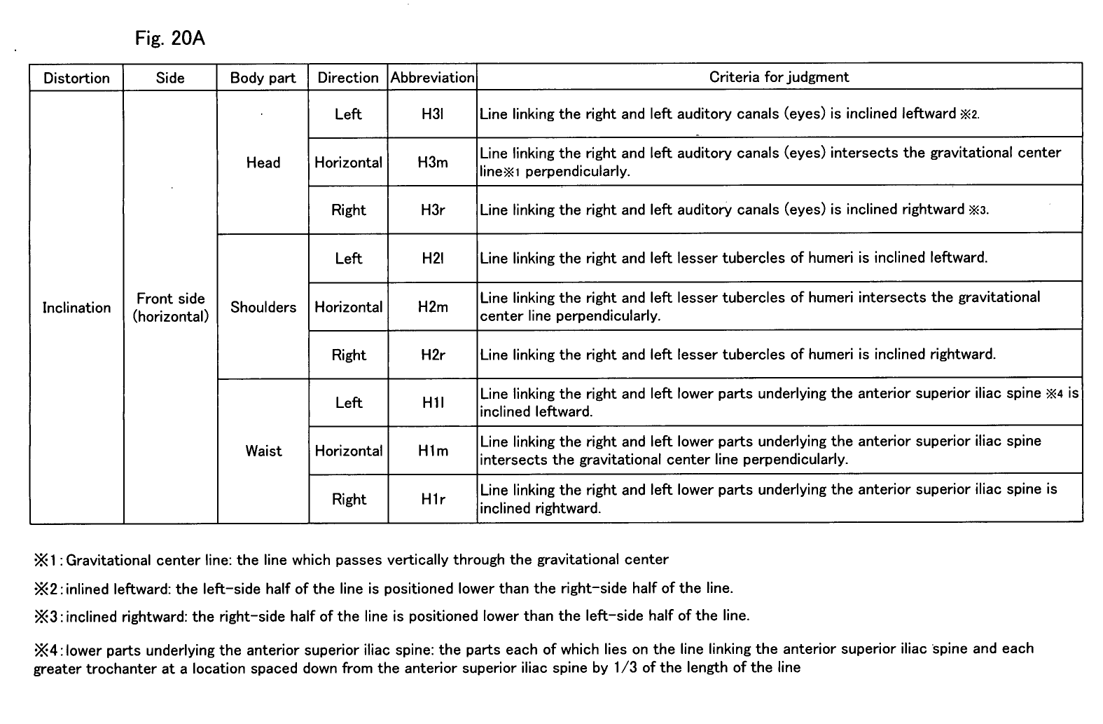

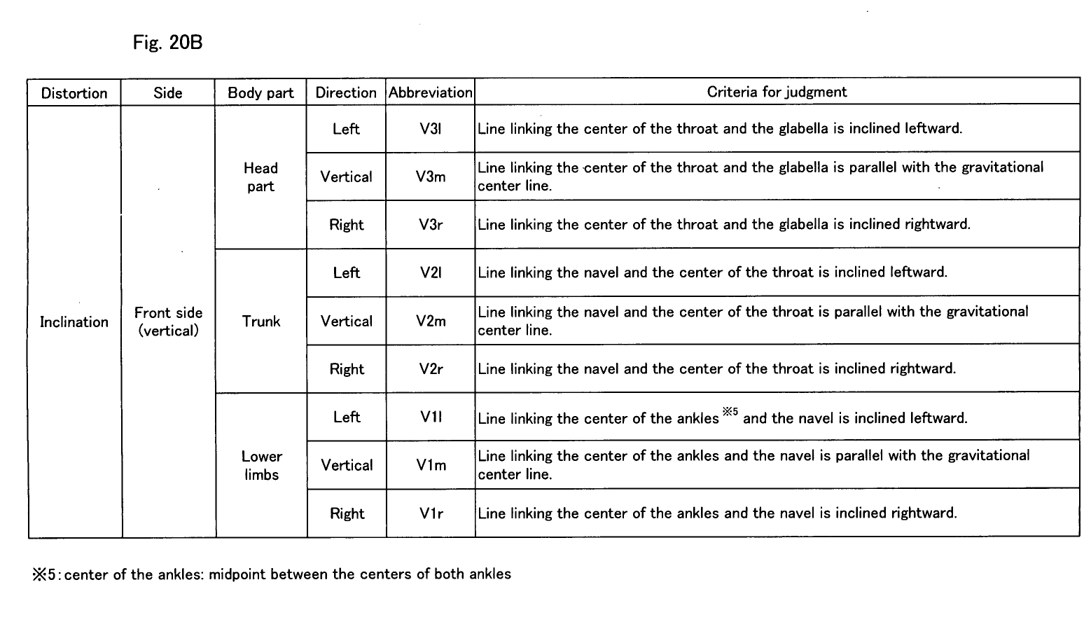

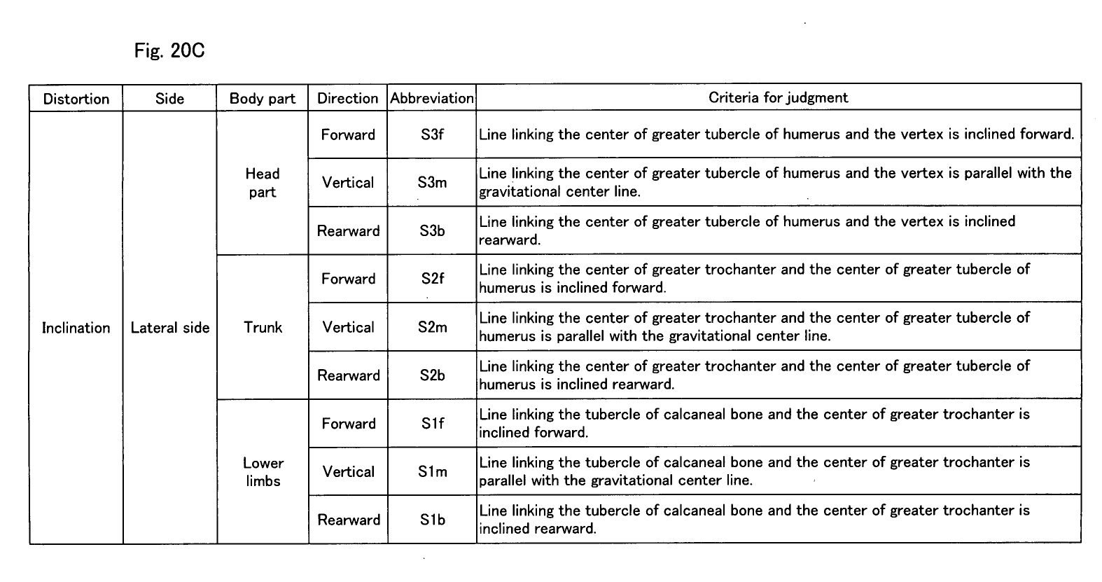

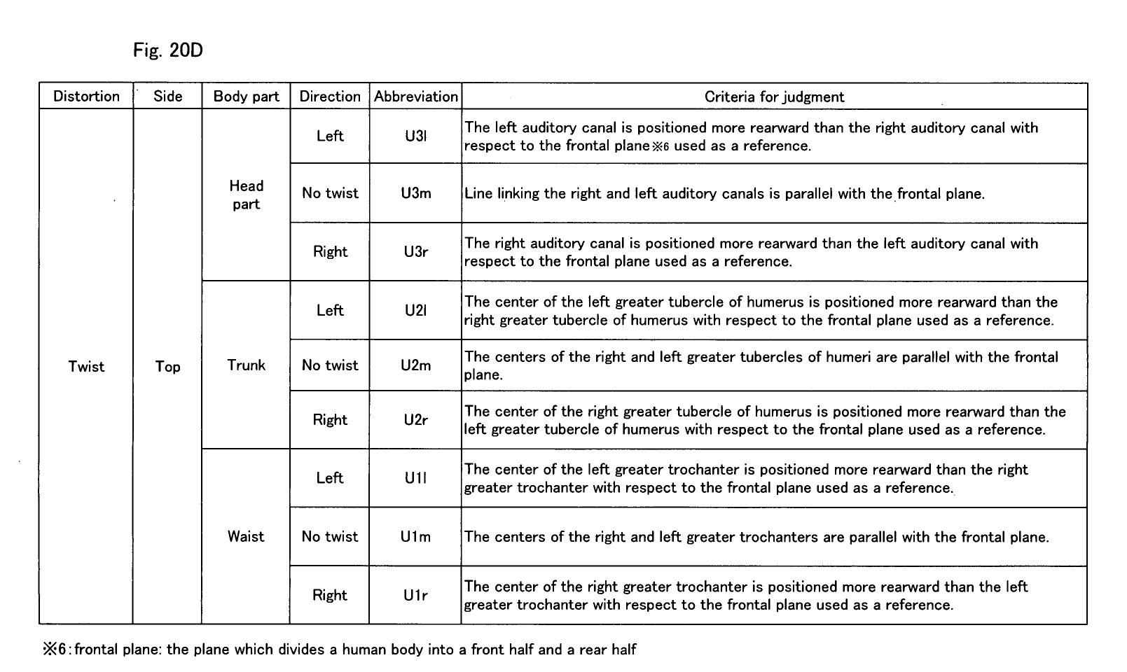

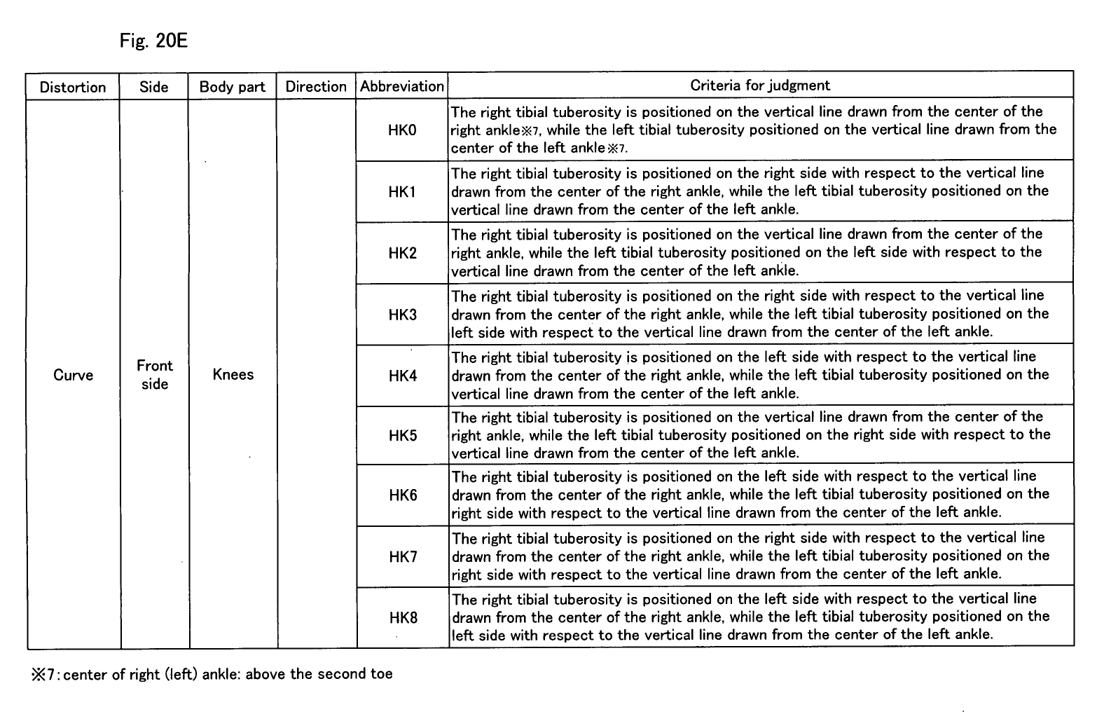

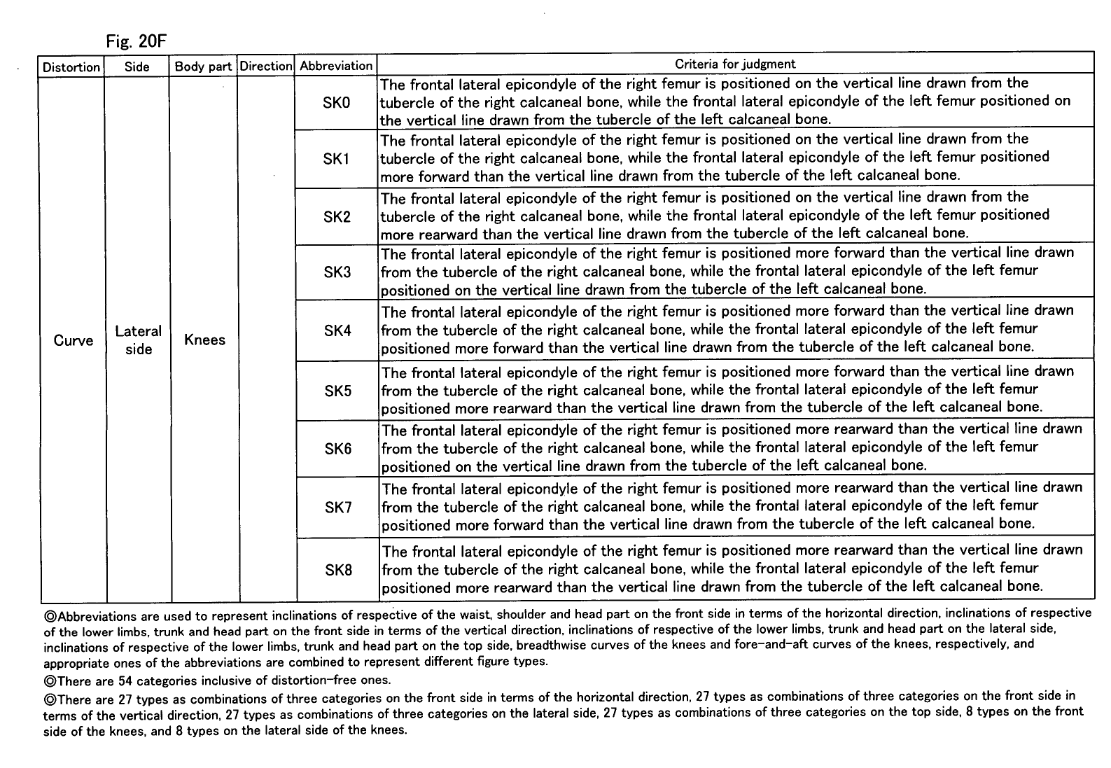

The figure judgment means 106 diagnoses and typifies the examinee's posture based on the coordinates of each posture diagnosis point received by the posture diagnosis point coordinates receiving means 105 and the gravitational center line calculated by the gravitational center line calculating means 104. FIGS. 20A, 20B, 20C, 20D, 20E and 20F show judgment criteria for typification of postures of examinees according to the program. Specific examples of figure judgment will be described with reference to this table. Reference is made to, for example, the case of judgment whether or not a posture of the examinee in the front-side photograph data as taken from the front side of the examinee is inclined relative to the horizontal axis. If the line segment linking the right and left auditory canals which are (part of) posture diagnosis points intersects the gravitational center line LW substantially perpendicularly, his head part is judged not to be inclined and its posture is categorized into a pattern identified with an identifier “H3m”. On the other hand, if the line segment is leftwardly inclined (that is, the left half of the line segment is positioned lower than the right half thereof), his head part is judged to be leftwardly inclined and its posture is categorized into a pattern identified with an identifier “H3l”. Alternatively, if the line segment is inclined rightwardly, his head part is judged to be rightwardly inclined and its posture is categorized into a pattern identified with an identifier “H3r”.

If the line segment linking the right and left lesser tubercles of humeri which are posture diagnosis points intersects the gravitational center line LW substantially perpendicularly, his shoulder part is judged not to be inclined and its posture is categorized into a pattern identified with an identifier “H2m”. On the other hand, if the line segment is leftwardly inclined, his shoulder part is judged to be leftwardly inclined and its posture is categorized into a pattern identified with an identifier “H2l”. Alternatively, if the line segment is inclined rightwardly, his shoulder part is judged to be rightwardly inclined and its posture is categorized into a pattern identified with an identifier “H2r”.

In the same manner as above, the posture of the examinee is typified as to other body parts and other photographing angles. Specifically, according to the program, the processor 1a gives an identifier (such as H3r or H2m mentioned above) to the posture of each body part photographed from each photographing angle to categorize the figure by reference to the coordinates of each posture diagnosis point specified relative to the photograph data and the gravitational center line LW contained in the photograph data. In this embodiment such identifiers are given as to identify figures of respective waist/shoulder/head parts inclined relative to the horizontal axis as viewed from the front-side angle, figures of respective lower limb/trunk/head parts inclined relative to the vertical axis as viewed from the front-side angle, figures of respective lower limb/trunk/head parts inclined relative to the vertical axis as viewed from a lateral-side angle, figures of respective lower limb/trunk/head parts twisted as viewed from the top-side angle, figures of the knee part curved breadthwise as viewed from the front-side angle, and figures of the knee part curved in the fore-and-aft direction as viewed from the lateral-side angle.

Since this embodiment does not have any photographing means to photograph the examinee from above, some contrivance is necessary to categorize the figure of the examinee as viewed from the top. For instance, a twisted position of the examinee's body can be diagnosed using as judgment material the distance between the gravitational center line LW contained in each of the front-side photograph data, left-side photograph data, right-side photograph data and rear-side photograph data and each of predetermined right and left posture diagnosis points forming a pair, for example, the centers of the right and left greater tubercles of humeri, which are specified in each of the photograph data items. This process includes the procedural steps of: determining the distances xl and xr from the centers of the right and left greater tubercles of humeri to the gravitational center line LW appearing in the front-side photograph data, the distance yl from the center of the left greater tubercle of humerus to the gravitational center line LW appearing in the left-side photograph data and the distance yr from the center of the right greater tubercle of humerus to the gravitational center line LW appearing in the right-side photograph data; and then calculating:
z=√ [(xl+xr)²+(yl+yr)²]
A=arctan [(yl+yr)/(xl+xr)].
These values each serve as a parameter indicative of the magnitude of a twist of the examinee's body about the body axis.

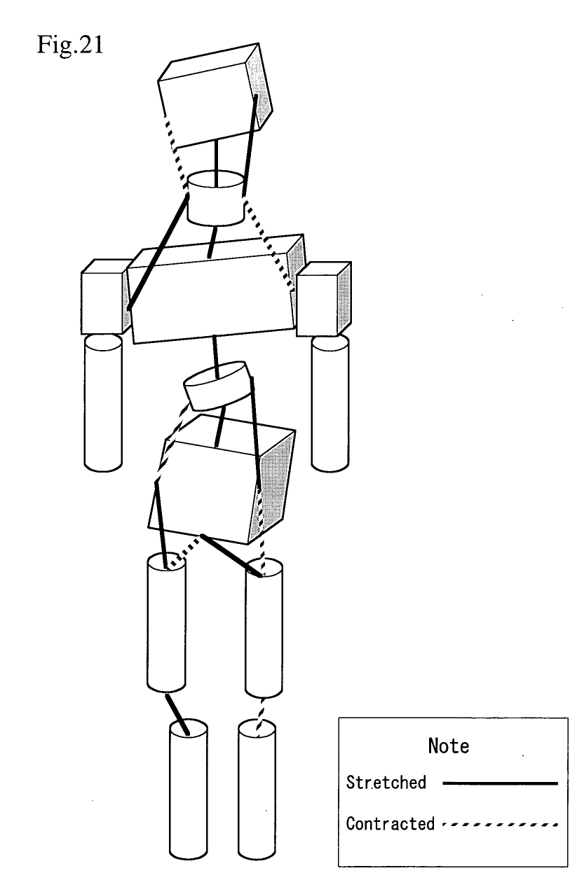

Further, the figure of the examinee is turned into a two- or three-dimensional model as shown in FIG. 21 according to the coordinate values of respective posture diagnosis points and identifiers given to categorize the postures of body parts of the examinee. Specifically, according to the program, the processor 1a turns the position of correlated ones (interconnected through muscle, tendon or the like in terms of human body structure for example) of posture diagnosis points into a model representing any one of a horizontal position, a vertical position, an inclined position (relative to the horizontal axis or vertical axis) and a twisted position (about the vertical axis.) Further, the processor 1a turns the condition of body tissue intervening between the correlated ones of the posture diagnosis points, such as epithelial tissue, supportive tissue, connective tissue, muscle tissue, nervous tissue, chest cavity, abdominal cavity, pelvic cavity, skeleton or contents, into a model expressing whether the body tissue is in a stretched condition or a contracted condition based on the distance between the correlated posture diagnosis points. These models are outputted in a manner to appeal to the vision of the diagnostician or the examinee as to whether or not the positional relation between the correlated posture diagnosis points is in a preferable condition, i.e., whether or not the posture of the examinee is in a good condition. For example, the centers of the greater tubercles of humeri, which are posture diagnosis points. forming a pair on the right and left sides of the examinee, are preferably positioned at equal height, and further, the line segment linking the two is preferably positioned substantially perpendicular to the median plane of the examinee (that is, the shoulder part of the examinee is not twisted.) If the two are positioned at equal height while the line segment linking the two positioned substantially perpendicular to the median plane of the examinee, the positional relation between these posture diagnosis points can be considered to be in a preferable condition. The model shown in FIG. 21 clearly expresses categories of such positional relation between posture diagnosis points.

The result of posture diagnosis by the figure judgment means 106 (including identifiers categorizing figures as described above and pattern models of figures of the examinee) is outputted in a manner to be displayed on the screen of the display 1e, printed out as a hard copy by the printer, transmitted to another computer connected to the equipment 1 via the communications interface 1g for communication, or stored in a predetermined storage area of the main memory 1b or auxiliary storage device 1c, or in a like manner.

Finally, the advice information output means 115 outputs information serving as advice about the posture of the examinee based on the typification of the posture of the examinee by the figure judgment means 106. For example, in accordance with posture categorizing identifiers given as related to the examinee's body there is outputted advice as stated below:
category identified by identifier “HK3”=> advice “Do exercise centered on the knee joints in addition to fundamental regular exercise.”;
category identified by identifier “Hlm2r3m”=> advice “Do exercise centered on the shoulder joints in addition to fundamental regular exercise.”;
category identified by identifier “V1r2m3m”=> advice “Do exercise centered on the hip joints in addition to fundamental regular exercise.”;
category identified by identifier “S1f2b3m”=> advice “Do exercise centered on the lumbosacral joint in addition to fundamental regular exercise.”
   Advice information database in which posture categorizing identifiers are connected to respective relevant advice items is previously stored in a predetermined storage area of the main memory 1b or auxiliary storage device 1c. The processor 1a searches the advice information database with a posture categorizing identifier used as a key and extracts the advice item to be outputted. The advice item is outputted in a manner to be displayed on the screen of the display 1e, printed out as a hard copy by the printer, transmitted to another computer connected to the equipment 1 via the communications interface 1g for communication, or stored in a predetermined storage area of the main memory 1b or auxiliary storage device 1c, or in a like manner.

It should be noted that the present invention is not limited to the foregoing embodiments having been described in detail. The specific feature of each part is not limited to the foregoing embodiments either and hence can be variously modified without departing from the concept of the present invention.

1. Posture diagnosis equipment capable of diagnosing a posture of an examinee for use in combination with a photographing device for photographing the posture of the examinee and a foot pressure measuring device for measuring a foot pressure of the examinee, the posture diagnosis equipment comprising:
photograph data receiving, means to receive photograph data obtained by photographing of the posture of the examinee from plural predetermined directions with the photographing device;
photograph data display means to display the photograph data received by the photograph data receiving means;
foot pressure data receiving means to receive foot pressure data obtained by measurement of the foot pressure of the examinee with the foot pressure measuring device;
gravitational center line calculating means to calculate a gravitational center line of the examinee which passes vertically through a gravitational center of the examinee based on the foot pressure data received by the foot pressure data receiving means;
posture diagnosis point coordinates receiving means to receive the coordinates of a posture diagnosis point serving as an indicator for diagnosis of the posture of the examinee, the posture diagnosis point being specified relative to the photograph data displayed by the photograph data display means; and
figure judgment means to diagnose and typify the posture of the examinee based on the gravitational center line calculated by the gravitational center line calculating means and the coordinates of the posture diagnosis point received by the posture diagnosis point coordinates receiving means.

2. The posture diagnosis equipment according to claim 1, wherein the photograph data receiving means is operative to receive plural photograph data items related to a single photographing direction,
the posture diagnosis equipment further comprising averaging means to sum up and then average the plural photograph data items related to the single photographing direction received by the photograph data receiving means to obtain a single photograph data item related to the single direction,
the photograph data display means being operative to display the photograph data having undergone the averaging process by the averaging means.

3. The posture diagnosis equipment according to claim 1, further comprising sharpening means to sharpen the photograph data, wherein
the photograph data display means is operative to display the photograph data having undergone the sharpening process by the sharpening means.

4. The posture diagnosis equipment according to claim 1, further comprising inclination error correction means to correct the photograph data to reduce an inclination error which is a degree of inclination of an upper edge of the foot pressure measuring device appearing in the photograph data relative to a horizontal axis of the photograph data, wherein the photograph data display means is operative to display the photograph data having undergone the correction process by the inclination error correction means.

5. The posture diagnosis equipment according to claim 4, wherein:
the foot pressure measuring device has inclination measurement reference points as a reference for measurement of the inclination error at predetermined locations thereon;
the photograph data to be received by the photograph data receiving means contains inclination measurement reference point data on the inclination measurement reference points photographed; and
the inclination error correction means is operative to measure the inclination error based on the inclination measurement reference point data contained in the photograph data and then reduce the inclination error.

6. The posture diagnosis equipment according to claim 5, wherein:
the foot pressure measuring device has plural inclination measurement reference points located on a horizontal straight line on a surface thereof facing the photographing device;
the photograph data to be received by the photograph data receiving means contains inclination measurement reference point data on the plural inclination measurement reference points photographed; and
the inclination error correction means is operative to reduce the inclination error, regarding as the inclination error a degree of inclination of a straight line linking the plural inclination measurement reference points appearing in the photograph data relative to the horizontal axis of the photograph data.

7. The posture diagnosis equipment according to claim 5, wherein:
the inclination measurement reference points are operative to blink;
the photograph data receiving means is operative to receive a photograph data item obtained when the inclination measurement reference points are bright and a photograph data item obtained when the inclination measurement reference points are dark; and
the inclination error correction means is operative to detect the inclination measurement reference points from the photograph data items.

8. The posture diagnosis equipment according to claim 1, further comprising gravitational center line display means to display the gravitational center line calculated by the gravitational center line calculating means in a manner to superimpose the gravitational center line on the photograph data displayed by the photograph data display means.

9. The posture diagnosis equipment according to claim 1, further comprising reference position determining means to determine a reference position based on a position which is applied with pressure sensed by the foot pressure measuring device when a reference member for use in determining the reference position is placed on the foot pressure measuring device.

10. The posture diagnosis equipment according to claim 9, further comprising horizontal distance calculating means to calculate a horizontal distance between the gravitational center line and a vertical center line passing through the reference position determined by the reference position determining means.

11. The posture diagnosis equipment according to claim 1, wherein the photograph data display means is capable of enlarged display of a region in the photograph data corresponding to a region around the posture diagnosis point when the posture diagnosis point coordinates receiving means receives the coordinates of the posture diagnosis point.

12. The posture diagnosis equipment according to claim 1, wherein the photograph data display means is capable of displaying information indicative of the name and approximate location of the posture diagnosis point when the posture diagnosis point coordinates receiving means receives the coordinates of the posture diagnosis point.

13. The posture diagnosis equipment according to claim 1, wherein the figure judgment means is capable of visually outputting the posture of the examinee typified with use of a two-dimensional or three-dimensional model.

14. The posture diagnosis equipment according to claim 13, wherein the figure judgment means is operative to categorize posture diagnosis points as to whether or not the posture diagnosis points are in preferable positions based on positional relation between correlated ones of the posture diagnosis points and then visualize the posture of the examinee in a manner to express resulting categories clearly.

15. The posture diagnosis equipment according to claim 13, wherein the figure judgment means is operative to visualize the posture of the examinee in a manner to express a stretched or contracted condition of body tissue intervening between correlated ones of the posture judgment points.

16. The posture diagnosis equipment according to claim 1, further comprising:
foot pressure typifying means to typify the foot pressure data received by the foot pressure data receiving means; and
foot pressure pattern display means to display a pattern of foot pressure typified by the foot pressure typifying means.

17. The posture diagnosis equipment according to claim 1, further comprising advice information output means to output information serving as advice about the posture of the examinee based on the posture of the examinee typified by the figure judgment means.

18. A program for use in the configuration of posture diagnosis equipment as recited in claim 1, which causes a computer to function at least as:
photograph data receiving means to receive photograph data obtained by photographing of a posture of an examinee from plural predetermined directions with a photographing device;
photograph data display means to display the photograph data received by the photograph data receiving means;
foot pressure data receiving means to receive foot pressure data obtained by measurement of a foot pressure of the examinee with a foot pressure measuring device;
gravitational center line calculating means to calculate a gravitational center line of the examinee which passes vertically through a gravitational center of the examinee based on the foot pressure data received by the foot pressure data receiving means;
posture diagnosis point coordinates receiving means to receive the coordinates of a posture diagnosis point serving as an indicator for diagnosis of the posture of the examinee, the posture diagnosis point being specified relative to the photograph data displayed by the photograph data display means; and
figure judgment means to diagnose and typify the posture of the examinee based on the gravitational center line calculated by the gravitational center line calculating means and the coordinates of the posture diagnosis point received by the posture diagnosis point coordinates receiving means.
